(19) United States
(12) Patent Application Publication
(10) Pub. No.: US 2026/0049105 A1
(43) Pub. Date: Feb. 19, 2026

(54) SELECTIVE MC4R LIGAND FOR TREATING OBESITY AND COGNITIVE LOSS

(71) Applicant: ARIZONA BOARD OF REGENTS ON BEHALF OF THE UNIVERSITY OF ARIZONA, Tucson, AZ (US)

(72) Inventors: Minying Cai, Tucson, AZ (US); Victor J. Hruby, Tucson, AZ (US)

(21) Appl. No.: 19/103,925

(22) PCT Filed: Aug. 17, 2023
(86) PCT No.: PCT/US23/72383

§ 371 Date: Feb. 14, 2025

Related U.S. Application Data
(60) Provisional application No. 63/371,819, filed on Aug. 18, 2022.

(51) Int. Cl. C07K 7/56; A61K 38/00

(52) U.S. Cl. CPC C07K 7/56; A61K 38/00

(57) ABSTRACT

Novel peptides having an extra Arg improve the melanocortin 4 receptor (MC4R) selectivity and potency are described herein. The peptides are used in compositions and methods for treating obesity and/or neurodegenerative diseases. A selective MC4R peptide agonist is administered to a subject in need of such treatment.

### CROSS-REFERENCES TO RELATED APPLICATIONS

This application claims benefit of U.S. Provisional Application No. 63/371,819 filed Aug. 18, 2022, the specification of which is incorporated herein in their entirety by reference.

### STATEMENT REGARDING FEDERALLY SPONSORED RESEARCH OR DEVELOPMENT

This invention was made with government support under Grant No. GM108040 awarded by National Institutes of Health. The government has certain rights in the invention.

### REFERENCE TO AN ELECTRONIC SEQUENCE LISTING

The contents of the electronic sequence listing (name of the file ARIZ 22_18 PCT.xml; Size: 59,642 bytes; and Date of Creation: Aug. 17, 2023) is herein incorporated by reference in its entirety.

### FIELD OF THE INVENTION

This present invention relates to a novel peptide, referred to herein as compound 9, which has been shown to be a super potent MC4R selective agonist with in vitro testing. This peptide can be used as a therapeutic for obesity and alleviate aging-induced decline of cognitive function.

### BACKGROUND OF THE INVENTION

Melanocortin 4 receptor (MC4R) is rhodopsin-like GPCR with multiple physiological functions. It spreads all over the human body but is centrally located in the hypothalamus. Previous research has found a wide variety of heterozygous loss-of-function mutations on MC4R cause a morbid early-onset obesity syndrome or hyperphagia, whereas gain-of-function mutations that increase receptor activity are associated with leanness. In this respect, MC4R has long been a hot pharmaceutical target for the treatment of obesity. Studies from the recent decade have shown that activation of MC4R via α-melanocyte stimulating hormone (α-MSH) happens after food intake and turns on downstream signaling which inflicts a neuronal impulse of satiety. Also, MC4R is highly related to energy homeostasis and expenditure which is dependent on its coupling to Kir7 upon specific ligand binding. Besides food intake control, MC4R is also found to play roles in controlling blood pressure, heart rate, and libido generation, making it a very important drug development target.

In recent decades, scientists have engaged in developing MC4R agonists. Even though many candidates were created, very few gave satisfying treatment to obesity for different reasons. Firstly, early problems come from the difficulty in making MC4R agonists selectively bind because melanocortin receptors (MCRs) have five different subtypes, and they resemble each other to a high degree. Even though in these five types, MC2R, which cannot be activated by MSHs and behaves more similarly to adrenergic receptors, is usually not functionally included in the melanocortin receptor family, the binding pocket characteristics of other four subtypes are still not easily distinguished, especially between MC3R and MC4R. Secondly, it was found in the recent decade that the canonical Gas-PKA pathway does not control the anticipated food intake. MCRs belong to membrane integrated GPCRs, whose biological functions depend highly on their dynamic conformations. Upon binding with different ligands, the MCRs' conformations will be induced to fit the bound ligands and thus changed to be more specific for recruiting different G proteins, which further trigger different downstream signaling. This phenomenon leads to the selection of different biological functions upon ligand binding. For example, THIQ is an MC4R selective small molecule agonist originally developed for appetite control in obese patients. However, animal studies found that it has little effect on appetite or inflammation but can strongly activate sexual activity. Another example is Melanotan II (MTII), which, even though it is not MC4R selective, shows strong MC4R activation potency with several effects, including increased moles and freckles, nausea, vomiting, loss of appetite, flushing of the face, involuntary stretching and yawning, and spontaneous erections. However, as an MC4R modified cyclic peptide molecule, Setmelanotide was FDA-approved in 2020 for its distinguished effect of no obvious cardiovascular side effects while maintaining promising appetite control during clinical trials. So, not all the MC4R agonists work equally to trigger a specific biological response. Thus numerous peptide/small molecule drugs for MCRs are still being developed so that anticipated control of specific biological function can be fulfilled and the mechanism of the control can be studied.

Here, the Inventors have designed and synthesized novel selective MC4R agonists based on an MTII template. Beta-amino acid, para-site halogenation on D-Phe, and the substitution of Nle to Arg were introduced. In particular, the present invention features a series of peptides 1-9 that are both novel and bioavailable. Analogue 9, among all the other derivatives, showed the best selectivity over hMC4R and excellent potency. These peptides can be used as bioavailable hMC4R selective melanotropins to target obesity and neurodegenerative diseases.

### BRIEF SUMMARY OF THE INVENTION

It is an objective of the present invention to provide peptides, compositions, and methods for treating obesity and neurodegenerative diseases, as specified in the independent claims. Embodiments of the invention are given in the dependent claims. Embodiments of the present invention can be freely combined with each other if they are not mutually exclusive.

In some embodiments, the present invention features a peptide according to any one of the following sequences:

(SEQ ID NO: 1)
Ac-Arg-cyclo[Asp-Pip-D-Phe-Arg-βTrp-Lys]-NH2;
(SEQ ID NO: 2)
Ac-Arg-cyclo[Asp-Pip-D-Phe-βArg-Trp-Lys]-NH2;
(SEQ ID NO: 3)
Ac-Arg-cyclo[Asp-Pip-D-Phe-βArg-βTrp-Lys]-NH2;
(SEQ ID NO: 4)
Ac-Arg-cyclo[Asp-Pip-D-Phe(4-F)-Arg-βTrp-Lys]-
NH2;
(SEQ ID NO: 5)
Ac-Arg-cyclo[Asp-Pip-D-Phe-(4-F)-βArg-Trp-Lys]-
NH2;
(SEQ ID NO: 6)
Ac-Arg-cyclo[Asp-Pip-D-Phe-(4-F)-βArg-βTrp-Lys]-
NH2;
(SEQ ID NO: 7)
Ac-Arg-cyclo[Asp-Pip-D-Phe-(4-Cl)-Arg-βTrp-Lys]-
NH2;
(SEQ ID NO: 8)
Ac-Arg-cyclo[Asp-Pip-D-Phe-(4-Cl)-βArg-Trp-Lys]-
NH2;
or
(SEQ ID NO: 9)
Ac-Arg-cyclo[Asp-Pip-D-Phe-(4-Cl)-βArg-βTrp-Lys]-
NH2.

In some embodiments, the present invention features a selective peptide agonist of melanocortin 4 receptor (MC4R). The peptide is according to the following sequence:

(SEQ ID NO: 9)
Ac-Arg-cyclo[Asp-Pip-D-Phe-(4-Cl)-βArg-βTrp-Lys]-
NH2.

In some embodiments, the MC4R peptide agonist is in a pharmaceutically acceptable carrier to form a pharmaceutical composition. In some embodiments, the peptide agonist is selective for MC4R.

In some embodiments, the pharmaceutical composition is effective for weight management, weight loss, or preventing or treating obesity or excess weight. In other embodiments, the pharmaceutical composition is effective for treating eating disorders, such as obesity.

In some other embodiments, the pharmaceutical composition is effective for preventing and treating a neurodegenerative disease. The neurodegenerative disease may be Alzheimer's disease, dementia, or other memory disorders, ataxia, amyotrophic lateral sclerosis, multiple sclerosis, Huntington's disease, Parkinson's disease, motor neuron disease, multiple system atrophy, or progressive supranuclear palsy. In other embodiments, the pharmaceutical composition is effective for stimulating or increasing neuromelanin production.

In some embodiments, the present invention features a method of treating obesity in a subject in need of such treatment. In other embodiments, the present invention features a method of treating an eating disorder in a subject in need of such treatment. In other embodiments, the present invention features a method of weight management in a subject in need thereof. The methods described herein may comprise administering to the subject a pharmaceutical composition comprising a therapeutically effective amount of a peptide. In preferred embodiments, the peptide is a peptide agonist of melanocortin 4 receptor (MC4R). In more preferred embodiments, the peptide agonist is selective for MC4R. In some embodiments, the peptide is according to the following sequence:

(SEQ ID NO: 9)
Ac-Arg-cyclo[Asp-Pip-D-Phe-(4-Cl)-βArg-βTrp-Lys]-
NH2.

In some embodiments, the present invention also features a method of preventing or treating a neurodegenerative disease in a subject in need thereof. In other embodiments, the present invention features a method of stimulating or increasing neuromelanin production in a subject in need thereof. The method may comprise administering to the subject a pharmaceutical composition comprising a therapeutically effective amount of a peptide. In preferred embodiments, the peptide is a peptide agonist of melanocortin 4 receptor (MC4R). In more preferred embodiments, the peptide agonist is selective for MC4R. In some embodiments, the peptide is according to the following sequence:

(SEQ ID NO: 9)
Ac-Arg-cyclo[Asp-Pip-D-Phe-(4-Cl)-βArg-BTrp-Lys]-
NH2.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A and 1B shows the PWR spectrum for hMC4R activated by different agonists. FIG. 1A shows spectra measured with light under p-polarization. FIG. 1B shows spectra measured with s-polarized laser light.

FIG. 2 is a comparison of cAMP production by compound 9 and MT-II. Cells were treated with one of six concentrations of compound 9 or MT-II (10⁻⁵M to 10⁻¹⁰M), and the cAMP produced was then measured indirectly through a competitive binding assay using radioactively labeled (H³) cAMP.

FIGS. 3A and 3B show molecular docking of compound 9 with hMC4R. FIG. 3A is an overview of compound 9's binding site on hMC4R. FIG. 3B is a close-up view of the various interactions compound 9 has in the binding pocket. Hydrogen bonds are shown as black dashed lines.

FIGS. 4A and 4B show compound 9 binding and interacting patterns generated via Molecular Dynamic (MD) simulation. FIG. 4A shows a representative image of the whole system obtained from MD simulation. Water molecules are shown as surface style, POPC molecules making bilayer. The hMC4R-analogue 9 complex is right inside the POPC bilayer. FIG. 4B shows a top view of the surface styled average structure of hMC4R-compound 9 complex obtained from 10 ns production run. compound 9 is shown in ball style and well fit into the binding pocket.

FIGS. 5A, 5B, and 5C shows magnified compound 9 binding and interacting patterns generated via Molecular Dynamic (MD) simulation. FIG. 5A shows the hydrogen bonding of compound 9 with key amino acids residing in the MC4R binding pocket. compound 9 is shown in the ball-stick model. FIG. 5B shows calcium ions (ball style) participate in connecting MC4R and compound 9; atoms with 5 angstroms from Calcium are shown in ball style. FIG. 5C shows the wobble switch L133 and W258 position after MD simulation with compound 9.

FIGS. 6A and 6B shows 2D NMR spectroscopy of compound 9. FIG. 6A shows the incorporation of TOCSY and ROESY spectra of compound 9. Labeling follows the numbering on compound 9 sequence: Ac-Arg1-cyclo[Asp2-Pip3-DPhe(4-Cl)4-sArg5-sTrp6-Lys7]-NH2. B: HN and HA protons in residue 5 are close to zero degrees, circled in a box.

### DETAILED DESCRIPTION OF THE INVENTION

Disclosed are various peptides, solvents, solutions, carriers, and/or components to be used to prepare compositions to be used within the methods disclosed herein. Also disclosed are the various steps, elements, amounts, routes of administration, symptoms, and/or treatments that are used or observed when performing the disclosed methods, as well as the methods themselves. These and other materials, steps, and/or elements are disclosed herein, and it is understood that when combinations, subsets, interactions, groups, etc. of these materials are disclosed, that while specific reference of each various individual and collective combination and permutation of these compounds may not be explicitly disclosed, each is specifically contemplated and described herein. It is also to be understood that the terminology used herein is for the purpose of describing particular embodiments only and is not intended to be limiting.

As used herein, the term “natural amino acids” refers to the twenty amino acids that are found in nature, i.e., occur naturally. The natural amino acids are as follows: alanine, arginine, glycine, asparagine, aspartic acid, cysteine, glutamine, glutamic acid, serine, threonine, histidine, lysine, methionine, proline, valine, isoleucine, leucine, tyrosine, tryptophan, and phenylalanine. This application adheres to the IUPAC rules of standard abbreviations for amino acids.

Each amino acid may be either natural or unnatural of the “D” or “L” configuration which corresponds to the stereochemical designation “S” and “R,” respectively, as defined in the RS system of Cahn et al., (Pure Applied Chemistry, 45:11-30 (1974), and references cited therein). As known to one of ordinary skill in the art, only L-amino acids are manufactured in cells and incorporated into proteins. As used herein, the letter “D” preceding any three-letter abbreviation for an amino acid, e.g., as in “D-Phe,” denotes the D-form of the amino acid, and a lack thereof refers to the L-form.

As used herein, the term “unnatural amino acids” refers to amino acids that are not naturally encoded or found in the genetic code of any organism. Typically, the unnatural amino acids are different from the twenty naturally occurring amino acids in their side chain functionality. Non-limiting examples of unnatural amino acids include 2-Naphthylalanine (Nal(2′)), Norleucine (Nle), and Pipecolic acid (Pip).

As defined herein, the term “agonist” refers to a compound that enhances a response. The agonist binds to the same site as the endogenous compound and produces the same type of signal, usually of equal or greater magnitude than the endogenous agent. As defined herein, the term “antagonist” refers to a compound that binds to the same site as the endogenous compound and diminishes or blocks the signal generated by the endogenous agent.

The peptide analogues of the present invention may be cyclized through bridging of the residues via ring closing reactions. As used herein, cyclization is denoted by “c” or “cyclo.” In some embodiments, the side chain of a residue is linked to the side chain of another residue via a linker. In some embodiments, the linker L₁ is a carba, lactam, disulfide, thioether, or succinic linker. As understood by one of ordinary skill in the art, the linker is not limited to the aforementioned examples and may depend upon the specific cyclization chemistry used to produce the cyclic peptide. As a non-limiting example, residues can be linked via an amide bond formation reaction, which may form a —(CH₂)—CO—NH—(CH₂)ₙ— bridge, where n=1,2,3,4. In addition, carbon-carbon bonds, lactone, thioether, ether, disulfide, and other covalent bonds can be used as a part of the ring closing reactions. Without wishing to limit the invention to a particular theory or mechanism, the type of linker can affect the structural, chemical, and biological activity of the peptide ligand.

As defined herein, a β-amino acid or β-peptide refers to an amino acid in which the amino group of —NH₂ is attached to the secondary carbon rather than the a carbon. For example, a methylene group (CH₂) is inserted into the side chain at the beta position of that side chain. The flexibility to generate a vast range of stereo- and regioisomers, together with the possibility of disubstitution, significantly expands the structural diversity of β-amino acids. For instance, the incorporation of β-amino acids has been successful in creating peptidomimetics that not only have potent biological activity, but are also resistant to proteolysis.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects.

Overweight and obesity involve abnormal or excessive fat accumulation that presents a risk to health. A body mass index (BMI) over 25 is considered overweight, and a BMI over 30 is considered obese. Obesity can lead to other diseases and health problems, such as heart disease, diabetes, high blood pressure, and certain cancers.

Neurodegenerative diseases are caused by the progressive deterioration of nervous system cells (neurons) in the brain and spinal cord. Non-limiting examples of neurodegenerative diseases include Alzheimer's disease, dementia, and other memory disorders, ataxia, amyotrophic lateral sclerosis, multiple sclerosis, Huntington's disease, Parkinson's disease, motor neuron disease, multiple system atrophy, and progressive supranuclear palsy. Symptoms of Neurodegenerative Disorders include but are not limited to, abnormal movements, blood pressure fluctuation, deteriorating and/or loss of memory and cognitive abilities, and problems with mobility, balance, swallowing, bladder and bowel function, sleep, breathing, heart function, mood, and speech.

As used herein, the terms “treat,” “treating,” or “treatment” refer to both therapeutic treatment and prophylactic or preventative measures, with the objective of preventing, reducing, slowing down (lessen), inhibiting, or eliminating an undesired physiological change, symptom, or disorder, such as the development or spread of obesity and/or neurodegenerative diseases. For purposes of this invention, beneficial or desired clinical results include, but are not limited to, alleviation of symptoms, diminishment of the extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disease as well as those prone to have the condition or disease or those in which the condition or disease is to be prevented or onset delayed. Optionally, the subject or patient may be identified (e.g., diagnosed) as one suffering from the disease or condition prior to administration of the compositions of the present invention. Subjects can be identified by, for example, any or a combination of appropriate diagnostic or prognostic assays known in the art.

A “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days, weekly, twice weekly, etc. Guidance can be found in the literature for appropriate dosages for given classes of pharmaceutical products.

The terms “administering” and “administration” refer to methods of providing a pharmaceutical preparation, composition, or formulation to a subject. The compositions described herein can be administered in a number of ways depending on whether local or systemic treatment is desired, and on the area to be treated. Such methods are well known to those skilled in the art and include, but are not limited to, administering the compositions orally, intranasally, parenterally (e.g., intravenously and subcutaneously), by intramuscular injection, by intraperitoneal injection, intrathecally, transdermally, extracorporeally, topically or the like.

For example, the peptide compositions described herein can be administered intranasally or administration by inhalant. As used herein, “intranasal administration” means delivery of the compositions into the nose and nasal passages through one or both of the nares and can comprise delivery by a spraying mechanism (device) or droplet mechanism (device), or through aerosolization of the composition, e.g., by using a nasal spray, atomizer, dropper, or syringe. Administration of the compositions by inhalant can be through the nose or mouth via delivery by a spraying or droplet mechanism. As used herein, “an inhaler” can be a spraying device or a droplet device for delivering the peptide composition, in a pharmaceutically acceptable carrier, to the nasal passages and the upper and/or lower respiratory tracts of a subject. Delivery can also be directly to any area of the respiratory system (e.g., lungs) via intratracheal intubation. A person of skill, monitoring a subject's clinical response, can adjust the frequency of administration and dosage of the medication according to methods known in the art.

Preparations for parenteral administration include sterile aqueous or non-aqueous solutions, suspensions, and emulsions. Examples of non-aqueous solvents are propylene glycol, polyethylene glycol, vegetable oils such as olive oil, fish oils, and injectable organic-esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Injectables can be prepared in conventional forms, either as liquid solutions or suspensions, solid forms suitable for solution of suspension in liquid prior to injection, or as emulsions. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, inert gasses and the like. Another approach for parenteral administration involves use of a slow release or sustained release system such that a constant dosage is maintained.

The exact amount of the compositions required will vary from subject to subject, depending on the species, age, weight, and general condition of the subject, the severity of the disorder being treated, the particular composition used, its mode of administration and the like. Thus, it is not possible to specify an exact amount for every composition. However, an appropriate amount can be determined by one of ordinary skill in the art using only routine experimentation given the teachings herein.

Pharmaceutical peptide compositions for topical or transdermal administration may include ointments, lotions, creams, gels, drops, adherent patches, iontophoresis, suppositories, sprays, liquids, and powders. Conventional pharmaceutical carriers, aqueous, powder or oily bases, thickeners, and the like may be necessary or desirable. A person of skill, monitoring a subject's clinical response, can adjust the frequency of administration and dosage of the medication according to methods known in the art.

In another aspect, the peptide compositions can be administered to a subject intramuscularly, e.g., by using muscular injections or electroporation. A person of skill, monitoring a subject's clinical response, can adjust the frequency of administration and dosage of the medication according to methods known in the art.

Pharmaceutical peptide compositions for oral administration include, but are not limited to, powders or granules, suspensions or solutions in water or non-aqueous media, pills, lozenges, capsules, sachets, or tablets. Thickeners, flavorings, diluents, emulsifiers, dispersing aids or binders may be desirable. A person of skill, monitoring a subject's clinical response, can adjust the frequency of administration and dosage of the medication according to methods known in the art.

As described above, the compositions can be administered to a subject in a pharmaceutically acceptable carrier. By “pharmaceutically acceptable,” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Typically, an appropriate amount of a pharmaceutically-acceptable salt is used in the formulation to render the formulation isotonic. Examples of the pharmaceutically-acceptable carrier include, but are not limited to, saline, Ringer's solution, and dextrose solution. The pH of the solution is preferably from about 5 to about 8, and more preferably from about 7 to about 7.5. Further carriers include sustained release preparations such as semi-permeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical compositions can include additional carriers, as well as thickeners, diluents, buffers, preservatives, surface active agents, and the like in addition to the compounds disclosed herein. Pharmaceutical compositions can also include one or more additional active ingredients such as antimicrobial agents, anti-inflammatory agents, anesthetics, and the like. Other suitable pharmaceutically acceptable carriers include solvents, dispersion media, coatings, isotonic and absorption delaying agents, and the like, which are compatible with pharmaceutical administration. Supplementary active compounds can also be incorporated into the compositions.

In some embodiments, the peptide compositions of the invention can be placed or stored in a container, bag, pack, or dispenser together with instructions for administration. For example, the instructions can include directions for administering the composition to the subject.

In some embodiments, the dosage can be administered to a subject once daily or in divided dosages throughout a day, depending on a subject's clinical response to the medication, as determined by methods known in the art. This dosage can be administered to a subject for one day, one a week, or a number of days, and then stopped if the subject responds immediately, or the dosage can be administered on a daily basis until a clinical response is noted. A person of skill can monitor a subject's clinical response to the administration of the peptide composition and administer additional dosages as needed. It is contemplated that the peptide composition can be administered to a subject on a daily basis, on an alternating daily basis, on a weekly basis, or at any interval in between.

It is advantageous to formulate the compositions in dosage units for ease of administration and uniformity of dosage. Dosage units refer to physically discrete units suited as unitary dosages for the subject to be treated, each unit containing a predetermined quantity of the peptide calculated to produce the desired therapeutic effect in association with the required pharmaceutical carrier.

Toxicity and therapeutic effects of the peptides can be determined by standard pharmaceutical procedures in cell cultures or experimental animals, e.g., for determining the LD₅₀ (the dose lethal to 50% of the population) and the ED₅₀ (the dose therapeutically effective in 50% of the population). Suitable animal models known in the art can be designed and used by one skilled in the art. The dose ratio between toxic and therapeutic effects is the therapeutic index, and it can be expressed as the ratio LD₅₀/ED₅₀. The peptides that exhibit high therapeutic indices are preferred. While peptides that exhibit toxic side effects may be used, care should be taken to design a delivery system that targets such compounds to the affected site in order to minimize potential damage to unaffected cells and thereby reduce side effects. Data obtained from the cell culture assays and animal studies can be used in formulating a range of dosages for use in humans. The dosage of the peptides lies preferably within a range of circulating concentrations that include the ED₅₀ with little to no toxicity.

The dosage may vary within this range depending upon the dosage form employed and the route of administration utilized. For any peptide used, the therapeutically effective dose can be estimated initially from cell culture assays in which, e.g., the rate of cell death is observed. A dose may be formulated in animal models to achieve a concentration range that includes the IC₅₀ (i.e., the concentration of the test compound which achieves a half-maximal inhibition of symptoms) as determined in cell culture. Such information may be used to more accurately determine useful doses in humans.

As used herein, the blood-brain barrier is made up of brain microvessel endothelial cells characterized by tight intercellular junctions, minimal pinocytic activity, and the absence of fenestra. These characteristics endow these cells with the ability to restrict passage of most small polar blood-borne molecules (e.g., neurotransmitter catecholamines, small peptides) and macromolecules (e.g., proteins) from the cerebrovascular circulation to the brain. The blood-brain barrier contains highly active enzyme systems as well, which further enhance the already very effective protective function. It is recognized that transport of molecules to the brain is not determined solely by molecular size but by the permeabilities governed by specific chemical characteristics of the permeating substance. Thus, besides molecular size and lipophilicity, the affinity of the substances to various blood proteins, specific enzymes in the blood, or the blood-brain barrier will considerably influence the amount of the drug reaching the brain.

In some embodiments, the pharmaceutical peptide compositions can be modified so that the peptide is able to cross the blood-brain barrier. A drug may be prepared by using a peptide that has been modified by cyclization, glycosylation, and/or methylation (e.g., N-methylation). An alternative method of modifying a drug is to prepare a redox system. To create a redox system, a drug is prepared by attaching the peptide to a carrier, such as a pyridinium carrier. Commonly used pyridinium carriers include substituted nicotinic acid and nicotinamide. After coupling, the drug-carrier complex is reduced, yielding a dihydropyridine. The reduced complex is then administered systemically. The reduced complex will cross the BBB due to its enhanced membrane permeability, and it will also be distributed elsewhere in the body.

According to some embodiments, the present invention features a peptide according to any one of the following sequences:

(SEQ ID NO: 1)
Ac-Arg-cyclo[Asp-Pip-D-Phe-Arg-BTrp-Lys]-NH2;
(SEQ ID NO: 2)
Ac-Arg-cyclo[Asp-Pip-D-Phe-βArg-Trp-Lys]-NH2;
(SEQ ID NO: 3)
Ac-Arg-cyclo[Asp-Pip-D-Phe-βArg-bTrp-Lys]-NH2;
(SEQ ID NO: 4)
Ac-Arg-cyclo[Asp-Pip-D-Phe(4-F)-Arg-BTrp-Lys]-
NH2;
(SEQ ID NO: 5)
Ac-Arg-cyclo[Asp-Pip-D-Phe(4-F)-βArg-Trp-Lys]-
NH2;
(SEQ ID NO: 6)
Ac-Arg-cyclo[Asp-Pip-D-Phe(4-F)-βArg-BTrp-Lys]-
NH2;
(SEQ ID NO: 7)
Ac-Arg-cyclo[Asp-Pip-D-Phe(4-Cl)-Arg-BTrp-Lys]-
NH2;
(SEQ ID NO: 8)
Ac-Arg-cyclo[Asp-Pip-D-Phe(4-Cl)-βArg-Trp-Lys]-
NH2;
or
(SEQ ID NO: 9)
Ac-Arg-cyclo[Asp-Pip-D-Phe(4-Cl)-βArg-BTrp-Lys]-
NH2.

According to other embodiments, the present invention features a selective peptide agonist of melanocortin 4 receptor (MC4R). The peptide is according to the following sequence:

(SEQ ID NO: 9)
Ac-Arg-cyclo[Asp-Pip-D-Phe-(4-Cl)-βArg-BTrp-Lys]-
NH2.

According to some other embodiments, the present invention features a pharmaceutical composition comprising a peptide agonist of melanocortin 4 receptor (MC4R) in a pharmaceutically acceptable carrier. The peptide is according to the following sequence:

(SEQ ID NO: 9)
Ac-Arg-cyclo[Asp-Pip-D-Phe-(4-Cl)-βArg-BTrp-Lys]-
NH2.

In some embodiments, the pharmaceutical composition is effective for weight management, weight loss, or preventing or treating obesity or excess weight. In other embodiments, the pharmaceutical composition is effective for treating eating disorders, such as obesity.

In some embodiments, the pharmaceutical composition is effective for preventing and treating a neurodegenerative disease. The neurodegenerative disease may be Alzheimer's disease, dementia, or other memory disorders, ataxia, amyotrophic lateral sclerosis, multiple sclerosis, Huntington's disease, Parkinson's disease, motor neuron disease, multiple system atrophy, or progressive supranuclear palsy.

In other embodiments, the pharmaceutical composition is effective for stimulating or increasing neuromelanin production.

In some embodiments, the peptide agonist is selective for MC4R. In other embodiments, the MC4R peptide agonist can cross the blood-brain barrier.

According to some embodiments, the present invention features a method of treating obesity in a subject in need of such treatment. The method may comprise administering to the subject a pharmaceutical composition comprising a therapeutically effective amount of a peptide. In preferred embodiments, the peptide is a peptide agonist of melanocortin 4 receptor (MC4R). In more preferred embodiments, the peptide agonist is selective for MC4R. In some embodiments, the peptide is according to the following sequence:

(SEQ ID NO: 9)
Ac-Arg-cyclo[Asp-Pip-D-Phe-(4-Cl)-βArg-BTrp-Lys]-
NH2.

According to other embodiments, the present invention features a method of treating an eating disorder in a subject in need of such treatment. The eating disorder may be obesity. In some aspects, the subject is overweight or obese. The method may comprise administering to the subject a pharmaceutical composition comprising a therapeutically effective amount of a peptide. In preferred embodiments, the peptide is a peptide agonist of melanocortin 4 receptor (MC4R). In more preferred embodiments, the peptide agonist is selective for MC4R. In some embodiments, the peptide is according to the following sequence:

(SEQ ID NO: 9)
Ac-Arg-cyclo[Asp-Pip-D-Phe-(4-Cl)-βArg-BTrp-Lys]-
NH2.

According to other embodiments, the present invention features a method of weight management in a subject in need thereof. The method may comprise administering to the subject a pharmaceutical composition comprising a therapeutically effective amount of a peptide. In preferred embodiments, the peptide is a peptide agonist of melanocortin 4 receptor (MC4R). In more preferred embodiments, the peptide agonist is selective for MC4R. In some embodiments, the peptide is according to the following sequence:

(SEQ ID NO: 9)
Ac-Arg-cyclo[Asp-Pip-D-Phe-(4-Cl)-βArg-BTrp-Lys]-
NH2.

According to some embodiments, the present invention features a method of preventing or treating a neurodegenerative disease in a subject in need thereof. The neurodegenerative disease is Alzheimer's disease, dementia, or other memory disorders, ataxia, amyotrophic lateral sclerosis, multiple sclerosis, Huntington's disease, Parkinson's disease, motor neuron disease, multiple system atrophy, or progressive supranuclear palsy. The method may comprise administering to the subject a pharmaceutical composition comprising a therapeutically effective amount of a peptide. In some aspects, the pharmaceutical composition is effective for stimulating or increasing neuromelanin production. In preferred embodiments, the peptide is a peptide agonist of melanocortin 4 receptor (MC4R). In more preferred embodiments, the peptide agonist is selective for MC4R. In some embodiments, the peptide is according to the following sequence:

(SEQ ID NO: 9)
Ac-Arg-cyclo[Asp-Pip-D-Phe-(4-Cl)-βArg-BTrp-Lys]-
NH2.

According to some other embodiments, the present invention features a method of stimulating or increasing neuromelanin production in a subject in need thereof. The method may comprise administering to the subject a pharmaceutical composition comprising a therapeutically effective amount of a peptide. In preferred embodiments, the peptide is a peptide agonist of melanocortin 4 receptor (MC4R). In more preferred embodiments, the peptide agonist is selective for MC4R. In some embodiments, the peptide is according to the following sequence:

(SEQ ID NO: 9)
Ac-Arg-cyclo[Asp-Pip-D-Phe-(4-Cl)-βArg-BTrp-Lys]-
NH2.

In some embodiments, the subject is a mammal. For example, the mammal is a human. In alternative embodiments, the mammal is non-human.

In conjunction with the methods described herein, the peptide may be administered in a dosage of about 0.001 mg/kg to 100 mg/kg of body weight. In some embodiments, the composition is administered at least once daily, weekly, or monthly. In some embodiments, the composition is administered intranasally, intravenously, subcutaneously, transdermally, or orally.

In some embodiments, the present invention features a pharmaceutical composition comprising a peptide agonist of melanocortin 4 receptor (MC4R) in a pharmaceutically acceptable carrier for use in a method of weight management in a subject in need thereof. In some embodiments, the peptide is according to the following sequence:

(SEQ ID NO: 9)
Ac-Arg-cyclo[Asp-Pip-D-Phe-(4-Cl)-βArg-BTrp-Lys]-
NH2.

In other embodiments, the present invention features a pharmaceutical composition comprising a peptide agonist of melanocortin 4 receptor (MC4R) in a pharmaceutically acceptable carrier for use in a method preventing or treating a neurodegenerative disease in a subject in need thereof. In some embodiments, the peptide is according to the following sequence:

(SEQ ID NO: 9)
Ac-Arg-cyclo[Asp-Pip-D-Phe-(4-Cl)-βArg-BTrp-Lys]-
NH2.

### Example

The following is a non-limiting example of the present invention. It is to be understood that said example is not intended to limit the present invention in any way. Equivalents or substitutes are within the scope of the present invention.

### Design of Novel MC4R Selective Ligands

Without wishing to limit the invention to a particular theory or mechanism, the positive charge of melanotropin plays an important role in the molecule interaction with MC4R. To increase the MC4R selectivity, an extra Arg was incorporated into the designed cyclized peptides (Table 1).

### Materials and Methods

### Design of Peptide Ligands

The design of hMC4R selective peptide started from the selection of an appropriate template which has ideal size for receptor binding pocket. All MCR subtypes share high similarities in their conformation thus the unique property of hMC4R needs to be put into consideration.

The peptides designed herein used the knowledge of MC4R structure and to generate modifications to the MTII (Ac-Nle-c[Asp-His-DPhe-Arg-Trp-Lys]-NH2 (SEQ ID NO: 10)) template. Even though MTII itself is not selective to MC4R, special modifications were introduced to improve the MC4R binding selectivity. For example, minor adjustments to the ring size were introduced so that the designed molecules still fit MC4R but not the other subtypes. In this respect, the ring size was varied with the normal Arg/Trp changed to either beta-homo-Trp, beta-homo-Arg, or both. Additionally, histidine was changed to a more rigid amino acid (e.g., Pro, Pip, Tic, Inp and Oic), so that the designed molecules are more fixed in specific shapes. Furthermore, including an Arg in the designed molecules provides the possibility for H-bonding and electrostatic interactions due to its sidechain. Lastly, adding bulky moiety at D-Phe site affects MC4R activating potency.

### Solid Phase Peptide Synthesis (SPPS)

The peptides were synthesized using Fmoc chemistry with an appropriate orthogonal protection strategy. N α-Fmoc-amino acids were obtained from Bachem, NovaBiochem, and Advanced ChemTech.

Sidechain protected amino acids include Fmoc-Trp(Boc)-OH, Fmoc-Arg(pbf)-OH and Fmoc-His(trt)-OH, so that the side chains can be removed during the final acidic cleavage step. Rink amide resin was purchased from Polymer Laboratories. Organic solvents and reagents were purchased from Aldrich and used without further purification. The first amino acid was added on the rink amide resin (loading rate: 0.39 mmol/g) after the Fmoc protection removal was completed under 3% piperidine and 2% DBU in DMF for 15 minutes twice. All peptides were coupled by the N-Fmoc solid-phase peptide strategy N—Fmoc amino acid (3 equiv), HCTU (3 equiv) in DMF with DIEA (3 equiv) added. The coupling happens in the filtered syringe shaken for 1 hour. Coupling completion was monitored by a Kaiser test. N terminal acetylation was done after coupling of final amino acid with 1:1:3(v/v) acetic anhydride:DIPEA:DCM for 20 minutes and repeated twice. In the sequence, Fmoc-Asp(allyl)-OH and Fmoc-Lys(alloc)-OH were applied so that their side-chain protection can be removed, and cyclization finished on resin without the interference of other side-chain protections. The allyl and alloc were removed under neutral conditions with catalytic amounts of Pd(PPh3)4 (26.6 equiv) in the presence of PhSiH3 (10 equi) and argon. After cyclization, cleavage of the peptide from the resin was finished in 3 hours with TFA cocktail (95:2.5:2.5 TFA:TIS:H2O v/v). The crude peptide obtained following cleavage from the resin showed a single major peak and purification was accomplished by semi-preparative C18 RP-HPLC (column: Vydac 218TP152022, 250/22 mm, 15-20 μm, 300 Å). The physicochemical properties and purity of the final peptides were assessed by ESI-MS, analytical C18 RP-HPLC (column: YMC-Pack ODS-AM 150_4.6 mm, S-3 μm, 120 Å). System 1: solvent A, 0.1% TFA in water; solvent B, 0.08% TFA in acetonitrile. System 2: solvent A, 1% formic acid in water; solvent B, 1% formic acid in methanol) and aqueous 0.1% TFA (v/v). The major peak of all compounds accounted for >95% of the combined total peak area monitored by a UV detector at 254 nM.

### Receptor Binding Assay

Competition binding experiments were performed on whole cells. The transfected HEK293 cell line with hMC4Rs were seeded on 96 well plates, 48 hours before the assay, 100,000 cells/well. For the assay, the medium was removed and cells were washed twice with a freshly prepared binding buffer containing 100% minimum essential medium with Earle's salt (MEM, GIBCO), 25 mM HEPES (pH 7.4), 0.2% bovine serum albumin, 1 mM 1,10-phenanthroline, 0.5 mg/L leupeptin, and 200 mg/L bacitracin. Cells were then incubated with different concentrations of unlabeled peptide and 125I-labeled [Nle4,D-Phe7]-α-MSH (PerkinElmer Life Science, 100,000 cpm/well, 0.1386 nM) for 40 minutes at 37° C. The medium was subsequently removed and each well was washed twice with the assay buffer. The cells are lysed by the addition of 250 μL of 0.1 NaOH and 250 μL of 1% Triton X-100. The lysed cell was transferred to the 12×75 mm glass tubes and the radioactivity was measured by Wallac 1470 WIZARD Gamma Counter. Data were analyzed using Graphpad Prism 6.0 (Graphpad Software, San Diego, CA).

### Adenylate Cyclase Assay

HEK293 cells transfected with human melanocortin receptors were grown to confluence in MEM medium (GIBCO) containing 10% fetal bovine serum, 100 units/mL penicillin and streptomycin, and 1 mM sodium pyruvate. The cells were seeded on 96 well plates 48 hours before the assay, (100,000 cells/well). For the assay, the medium was removed and cells were rinsed with 1 mL of MEM buffer (GIBCO) or with Earle's balanced salt solution (EBSS, GIBCO). An aliquot (0.4 mL) of Earle's balanced salt solution was placed in each well along with isobutylmethylxanthine (IBMX; 5 μL; 0.5 mM) for 1 minute at 37° C. Varying concentrations of melanotropins (0.1 mL) were added and the cells incubated for 3 minutes at 37° C. The reaction was stopped by aspirating the buffer and adding ice-cold Tris/EDTA buffer to each well (0.15 mL). The 96 well plates were covered and placed on ice. After dislodging the cells, the plates were placed in a boiling water bath for 15 minutes. The cell lysate was then centrifuged for 10 minutes (4000 rpm), and 50 μL of the supernatant was aliquoted into perkinElmer glass fiber filter plate. cAMP content was measured by competitive binding assay in addition to 50 μL H³ cAMP and 100 μL PKA. Then the system was placed on ice for 2 hours and then vacuum and filtered. 100 μL of the OptiPhase Supermix scintillation fluid was added to the plate. The plates stayed for 8 hours and then read by MicroBeta radiation plate reader.

### Plasmon Waveguide Resonance(PWR) Spectrum

PWR uses polarized light generated from a CW He—Ne laser (λ=632.8 nm or λ=543.5 nm), which passes through a glass prism under total internal reflection conditions. A thin metal film (Ag), which is deposited on the external surface of the prism, is overcoated with another thin layer of SiO2 as dielectric media. When there is an object attached to the dielectric layer of SiO2, and polarized laser forms a specific incident angle with the outer surface layer, the resonant excitation of collective electronic oscillations (plasmons) happens and generates an evanescent electromagnetic field localized at the dielectric film, which gives the absorption spectrum at that specific angle. This can be used to probe the optical properties of molecules immobilized on the outer surface of the prism. Resonance is achieved either by varying the laser light wavelength at a fixed angle or by varying the angle at a fixed laser wavelength(we used the latter one in this research). Any changes of the immobilized yield distinct incident angle that evanescence happens in the dielectric layer. Due to the ability that laser light can be polarized to either parallel (p) or perpendicular (s) to the incident plane, characterization of the molecular organization of anisotropic systems can be fulfilled, such as biomembranes containing integral proteins. Under the experimental conditions described herein, the optical parameters obtained with the p-polarization refer to the perpendicular direction, and those obtained with s-polarization refer to the parallel direction, relative to the bilayer membrane surface.

Resonance spectra in this study were obtained using a PWR instrument from Biopeptek Pharmaceuticals LLC. (Malvern, PA). Recombinant HEK293 cells expressing the hMC4R were grown to confluency. The confluent cell monolayers were then washed with Ca²⁺ and Mg²⁺-deficient phosphate-buffered saline and were harvested in the same buffer containing 0.02% ethylenediaminetetraacetic acid (EDTA). After centrifugation at 1500 g for 10 minutes, the cells were homogenized in an ice-cold 10 mM Tris-HCl buffer and 1 mM EDTA (pH 7.4). A crude membrane fraction was collected by centrifugation at 40,000 g for 20 minutes at −4° C. and was stored at −80° C. until use. Before the PWR experiment, the membrane fraction was resuspended in a 20 mM Tris-HCl buffer (pH 7.4) by mild homogenization. Protein concentration was determined by using a Bradford assay. The agonists NDP-α-MSH, MTII, RM493 used in the PWR experiments were synthesized in the method as described above.

### Assay Data Analysis

IC50 and EC50 values represent the mean of duplicate experiments performed in triplicate. IC50 and EC50 estimates and their associated standard errors were determined by fitting the data using nonlinear least-squares analysis, with the help of Graphpad Prism 6.0 (Graphpad Software, San Diego, CA).

### Preparation of Protein and Ligand for Docking

Conformational based design of selective MC4R follows the NMR structure of MTII, SHU9119. The MC4R receptor model used for docking was the one in-silico model created by superimposing the MC4R sequence on the solved structure of rhodopsin. All docking experiments were carried out with the program Maestro. First, the ligand was prepared for docking by running the program LigandPrep. Then, Grid Preparation was used to generate the docking site on the receptor. Finally, the docking protocol in Maestro was used to carry out the pairing between the receptor and peptide. Files were exported in PDB format, and images were generated with Pymol.

In brief, the hMC4R structure PDB: 7AUE was obtained and imported to Maestro (Schrodinger Maestro, Release 2021-1; Schrodinger: New York, NY, 2021) and prepared using Schrodinger's Protein Preparation Wizard28. The G protein complex was removed and only MC4R was kept. Then the protein was subjected to energy minimization using Schrodinger implementation of OPLS3 force field. Peptide structures were built into extended structures with standard bond lengths and angles, and they were minimized using the OPLS3 force field and the Polak-Ribier conjugate gradient (PRCG). Optimizations were converged to a gradient rmsd of less than 0.05 kJ/A mol or continued until a limit of 50,000 iterations was reached. Aqueous solution conditions were simulated using the continuum dielectric water solvent model (GB/SA). Extended cutoff distances were defined at 8 Å for van der Waals, 20 Å for electrostatics, and 4 Å for H-bonds. Conformational profiles of the cyclic peptides were investigated by the hybrid Monte Carlo/low frequency mode (MCMM/LMCS) procedure as implemented in MacroModel using the energy minimization parameters as described above. MCMM torsional variations and low mode parameters were set up automatically within Maestro graphical user interface. A total of 20,000 search steps were performed, and the conformations with an energy difference of 50 kJ/mol from the global minimum were saved. Interatomic dihedral angles were measured for each peptide analogue using the Maestro graphical user interface.

### Molecular Docking

Before docking the ligand, MC4R ligand binding site was identified using Schrödinger SiteMap 4.129. Binding sites with at least 15 site points were generated using the fine grid option. Next step was grid generation: A grid box with a specific dimension was generated to limit the area of ligand docking calculation. A grid box size of 15×15×15 Å was centered according to the sitemap plots of the binding site. The receptor grid was defined as an enclosed box at the centroid of the ligand. Finally, docking was done with Schrödinger Glide. A flexible docking calculation with standard precision (SP) Glide algorithm was performed and after the post docking minimization, we used the pose with the best docking score for molecular dynamics simulation

### Molecular Dynamics(MD) Simulation

The initial configuration of the full system used in the study was prepared. MC4R (modified 7AUE) docked with ligands was placed into 2 boxes of 1-palmitoyl2-oleoylphosphatidylcholine (POPC) layer which has the aliphatic parts inwards to make the biomembrane bilayer according to the orientation in the OPM database. Two water boxes were placed above and below the lipid bilayer to complete the structure. The initial structure prepared above was simulated using NAMD version 2.931 Force field parameters for ligands. The initial system was minimized for 30,000 steps. The simulation cell size of the original box was maintained, with periodic boundary condition (PBC). The particle mesh Ewald (PME) method was used to calculate long-range electrostatic interactions, and the van der Waals interactions were cut off at 12 Å. The PME grid spacing was 1.0 Å, and the tolerance was 10-6. The SHAKE algorithm constrained hydrogen length with a tolerance of 1.0-8 Å. The minimization system was heated at a rate of 1.5 K/ps to 300 K. The full system was then equilibrated for 5 ns in an isothermal-isobaric ensemble, with a Langevin piston used to maintain a constant pressure at 1 atm, and a Langevin temperature control maintaining a constant temperature of 300 K36. Finally, the equilibrated system was simulated for 10 ns and the trajectories were used for analysis. The simulation systems compromised a box of 150 Å×150 Å×164 Å. The computations were carried out on the University of Arizona high-performance computing facility utilizing GPU-equipped nodes.

1D and 2D NMR Spectrometry with Temperature Variation

Peptide concentration for the NMR experiments was 6 mM. The peptide sample was prepared in 0.6 mL aqueous acetate buffer (50 mM CD3COONa, 1 mM NaN3, 10% v/v D2O). The pH of the sample was adjusted to 4.5 by using DCI as necessary. Variable temperature 1H NMR spectra were recorded on a Bruker DRX-500 spectrometer equipped with BBO probe. 1D proton spectra were collected at 288 K, 293 K, 298 K and 303 K with 3-9-19 Watergate solvent suppression. 2D NMR spectra were recorded on a Bruker Avance NEO-800 spectrometer equipped with an inverse TCl cryoprobe. 2D NMR spectra (TOCSY, ROESY, NOESY) were acquired at 298 K using excitation sculpting to suppress the solvent signal. TOCSY used 80 ms DIPSI-2 spin-lock at 10 kHz, ROESY used 200 ms CW spin-lock at 3.5 kHz, and NOESY used a 350 ms mixing time. Relaxation delay for all 2D experiments was set to 1.5 seconds. All 2D data were recorded with 1k complex points in the directly observed dimension and 128 of 512 complex pairs in the indirect dimension using non-uniform sampling (NUS) with a 25% Poisson gap schedule. NUS Data were reconstructed using MIST algorithm in MNova (0.75 threshold, 100 iterations). J-coupling for backbone amide protons (³J_{HN-HA}) was measured from a 1D proton spectrum recorded at 800 MHz.

### Results

When comparing the binding properties of all these 9 analogues for 4 MCR subtypes, the 24 membered ring is fairly favored by hMC1R where analogue 3, 6 and 9 showed lower binding affinities than the others. However both 24 and 25 membered rings exhibit better binding affinity than that on hMC3R and hMC5R. However due to hMC1R's not possessing as much as acidic amino acids at its key binding area none of these 9 analogues show impressive hMC1R activating potencies. And it is also shown that none of the 9 analogues exhibit impressive activating potencies on both hMC3R and hMC5R, indicating that the strategies of adding positive charge and adjusting ring size did take effect in adding hMC4R selectivity. Within hMC4R, almost all analogues with single extra carbon on backbone showed no satisfying potencies but analogue 3 and 9 performs both binding affinity and activating potency in an expected range. Analogue 9 shows the most improved potency and selectivity as it activates hMC4R (IC50=0.4 nM, EC50=4.2 nM). This is about 68 times higher in binding affinity to hMC4R than hMC1R, and over 1000 times higher than that on hMC3R and hMC5R. Apart from analogue 9, compound 3 also shows excellent binding affinity on hMC4R, but it also behaves similarly on hMC3R (IC50=1 nM on hMC4R and IC50=7 nM of hMC3R). All detailed activity data for 9 compounds is shown in table 1 and table 2.

TABLE 1
Amino acid sequence of the nine novel compounds described herein. For reference, the sequence of
MT-II (e.g., SEQ ID NO: 10) is also provided as a control. Ac=acetylated; B = beta amino acid.
Competitive Binding Assay and cAMP Assay Results of 9 Compounds for hM4CR. Note: the sequence
ID number is equivalent to the compound/analogue number used herein.

| | | hM4CR | | | |
|---|---|---|---|---|---|
| SEQ | | IC50 | | EC50 | |
| ID NO: | Sequence | (nM) | BE% | (nM) | Act% |
| 1 | Ac-Arg-cyclo[Asp-Pip-D-Phe-Arg-BTrp-Lys]-NH2 | NA | 0 | >1000 | 63 |
| 2 | Ac-Arg-cyclo[Asp-Pip-D-Phe-βArg-Trp-Lys]-NH2 | 0.4 | 100 | 47 ± 24 | 67 |
| 3 | Ac-Arg-cyclo[Asp-Pip-D-Phe-βArg-BTrp-Lys]-NH2 | 0.1 | 100 | 16 ± 4 | 84 |
| 4 | Ac-Arg-cyclo[Asp-Pip-D-Phe(4-F)-Arg-BTrp-Lys]-NH2 | >1000 | 9 | 630 ± 175 | 46 |
| 5 | Ac-Arg-cyclo[Asp-Pip-D-Phe(4-F)-βArg-Trp-Lys]-NH2 | >1000 | 23 | >1000 | 84 |
| 6 | Ac-Arg-cyclo[Asp-Pip-D-Phe(4-F)-βArg-BTrp-Lys]-NH2 | 248 ± 17 | 59 | >1000 | 120 |
| 7 | Ac-Arg-cyclo[Asp-Pip-D-Phe(4-Cl)-Arg-BTrp-Lys]-NH2 | 10 | 100 | 240 ± 10 | 57 |
| 8 | Ac-Arg-cyclo[Asp-Pip-D-Phe(4-Cl)-βArg-Trp-Lys]-NH2 | 8 | 100 | 110 ± 90 | 66 |
| 9 | Ac-Arg-cyclo[Asp-Pip-D-Phe(4-Cl)-βArg-BTrp-Lys]-NH2 | 0.4 | 100 | 4 ± 2 | 87 |
| 10 | Ac-Nle-cyclo[Asp-His-D-Phe-Arg-Trp-Lys]-NH2 | 1.4 | 100 | 10 | 100 |

IC50 is the concentration of peptide at 50% specific binding (N=4). % BE is the percentage of [125I]NDP-α-MSH displacement at 10 μM. NB means that 0% of [125I]NDP-α-MSH displacement was observed at 10 μM. EC50 is the effective concentration of peptide that could generate 50% maximal intracellular cAMP accumulation (N=4). % max effect is the percentage of cAMP produced at a ligand concentration of 10 μM, in relation to MT-II. NA indicates 0% cAMP accumulation observed at 10 μM. The peptides were tested over a range of concentrations from 10-10 to 10-5 M.

TABLE 2
Competitive Binding Assay and cAMP Assay Results of 9 Compounds for hM1CR, hM3CR, and hM5CR.

| SEQ | hM1CR | | | | hM3CR | | | | hM5CR | | | |
|---|---|---|---|---|---|---|---|---|---|---|---|---|
| ID | IC50 | BE | EC50 | Act | IC50 | BE | EC50 | Act | IC50 | BE | EC50 | Act |
| NO: | (nM) | % | (nM) | % | (nM) | % | (nM) | % | (nM) | % | (nM) | % |
| 1 | 3 ∓ 1 | 100 | 120 ∓ 24 | 88 | 149 ∓ 13 | 60 | 680 ∓ 25 | 88 | 350 ∓ 22 | 66 | 580 ∓ 340 | 95 |
| 2 | 0.1 | 100 | 220 ∓ 70 | 120 | 600 ∓ 23 | 24 | >1000 | 89 | NA | 0 | >1000 | 90 |
| 3 | 270 ∓ 10 | 30 | 190 ∓ 90 | 82 | 7 ∓ 1 | 100 | 120 ∓ 10 | 87 | 607 ∓ 13 | 46 | 290 ∓ 50 | 82 |
| 4 | 0.2 | 100 | 64 ∓ 10 | 90 | 117 ∓ 5 | 100 | 450 ∓ 830 | 76 | 47 ∓ 3 | 100 | 530 ∓ 65 | 72 |
| 5 | 14 ∓ 2 | 100 | 340 ∓ 0 | 100 | 59 | 100 | >1000 | 59 | NA | 0 | >1000 | 62 |
| 6 | NA | 0 | >1000 | 61 | 396 ∓ 12 | 39 | >1000 | 49 | 59 ∓ 6 | 90 | >1000 | 51 |
| 7 | 0.9 | 100 | 99 ∓ 0 | 67 | 37 ∓ 2 | 70 | >1000 | 24 | NA | 0 | >1000 | 21 |
| 8 | 5 ∓ 1 | 100 | 130 ∓ 0 | 89 | 108 ∓ 5 | 100 | 360 ∓ 25 | 46 | NA | 0 | >1000 | 38 |
| 9 | 27 | 100 | 240 ∓ 11 | 90 | >1000 | 10 | >1000 | 63 | 70 ∓ 5 | 75 | 295 ∓ 165 | 76 |
| 10 | 7 | 100 | 32 | 100 | 9 | 100 | 3 | 100 | 6 | 100 | 2 | 100 |

Note:
the sequence ID number is equivalent to the compound/analogue number used herein.

IC50 is the concentration of peptide at 50% specific binding (N=4). % BE is the percentage of NDP-α-MSH displacement at 10 μM. NB means that 0% of [125I]NDP-α-MSH displacement was observed at 10 μM. EC50 is the effective concentration of peptide that could generate 50% maximal intracellular cAMP accumulation (N=4). % max effect is the percentage of cAMP produced at a ligand concentration of 10 μM, in relation to MT-II. NA indicates 0% cAMP accumulation observed at 10 μM. The peptides were tested over a range of concentrations from 10-10 to 10-5 M.

The results of the cAMP activity assays are summarized in Tables 1 and 2. For comparison, the values produced by MT-II as a control are also reported. Receptor activation was assessed by comparing the EC₅₀ of a compound for each receptor. The EC₅₀ is the concentration of drug necessary to induce 50% of maximum activity. For the three non-halogenated peptides (compounds 1-3), no selectivity for hMC4R was observed. When fluorine was placed on the Phe residue (compounds 4-6), activation of all receptors was negatively impacted. In particular, compound 6 produced an EC₅₀ greater than 1000 nM at each receptor. Halogenation of Phe with chlorine (compounds 7-9) improved receptor activation, especially of hMC1R and hMC4R. Additionally, the incorporation of β-amino acids produced a peculiar pattern for MC4R. For the compounds with chlorine, activation of MC4R was strongest when both β-Arg and β-Trp were used and weakest when only β-Trp was used. This pattern was also observed for the compounds that were not halogenated.

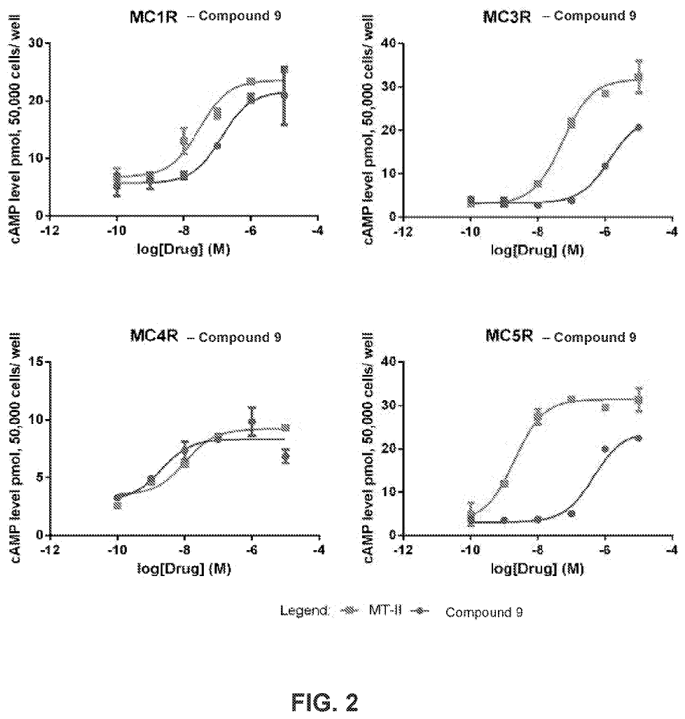

Ultimately, compound 9 was discovered to be a selective agonist of hMC4R with an EC₅₀ of 4 nM. This EC₅₀ was 60 to >260-fold smaller than the EC₅₀ of any other receptor. FIG. 2 shows a comparison dose-responsive curve of compound 9 with the MTII cAMP activity.

### Peptide Purification and Characterization

All 9 compounds were synthesized manually through the use of Fmoc chemistry. Once synthesis was complete, the peptides were cleaved, acetylated, and cyclized. Peptides were purified by HPLC and characterized by mass spectrometry. The sequences of all the compounds are in Table 1, and the physiochemical properties are in Table 3.

TABLE 3
Mass Spectrometric analysis of the compounds described
herein. Mass Spectrometry was performed with a Bruker
Amazon Ion Trap at survey mass resolution level and
ESI positive ion mode ionization. RP-HPLC was performed
on Shimadzu SCL-10A HPLC on a C18-bonded silica gel
column (Vydac 218TP1010, 1.0 × 25 cm) by eluting
2% to 80% of acetonitrile (0.1% TFA) in Nano pure water
(0.1% TFA) over 30 min with a flow rate of 3 mL/min.

| | Molecular | | | HPLC |
|---|---|---|---|---|
| SEQ | Weight | m/z [M + 2H]²⁺ | m/z [M + 2H]²⁺ | Retention |
| ID NO: | (g/mol) | Expected | Observed | Time (min) |
| 1 | 1055.26 | 529.63 | 528.33 | 11.41 |
| 2 | 1055.26 | 529.63 | 528.31 | 11.52 |
| 3 | 1069.28 | 536.64 | 535.32 | 11.54 |
| 4 | 1073.25 | 538.63 | 537.32 | 11.48 |
| 5 | 1073.25 | 538.63 | 527.32 | 11.50 |
| 6 | 1087.27 | 545.64 | 544.32 | 11.49 |
| 7 | 1089.70 | 546.85 | 545.31 | 11.35 |
| 8 | 1089.70 | 546.85 | 545.30 | 11.33 |
| 9 | 1103.73 | 553.87 | 552.33 | 11.39 |

PWR Analysis of hMC4R Structural Change Upon Peptide Analogue 9 Binding

MCRs belong to GPCR, which function is highly dependent on their conformations at activated state. Binding with different ligands leads to different GPCR activated conformation, which further inflicts different binding potentials between Ga subtypes. Binding differently with Gai, Gas, Gaq, etc. triggers different biological signaling and functions. Tremendous studies have elucidated relations of MC4R biased signaling. In brief, appetite control is related to the Gaq-PLC pathway while blood pressure increase is distinctly related to the Gas-PKA pathway. RM493 (a Gaq biased agonist) and THIQ (a Gas biased agonist) were analyzed to be compared because of their distinct effect of appetite control and blood pressure increase separately. At the same time, NDP-α-MSH and MTII were applied as a control for their function as a balanced agonist on hMC4R, and for their similar ability to equally activate appetite control and blood pressure increase. Under the experimental conditions employed herein, the oscillation of light with p-polarization reflects the dimension of objects perpendicular to the membrane, and those obtained under the s-polarization vector refer to the dimension in parallel with the bilayer membrane surface.

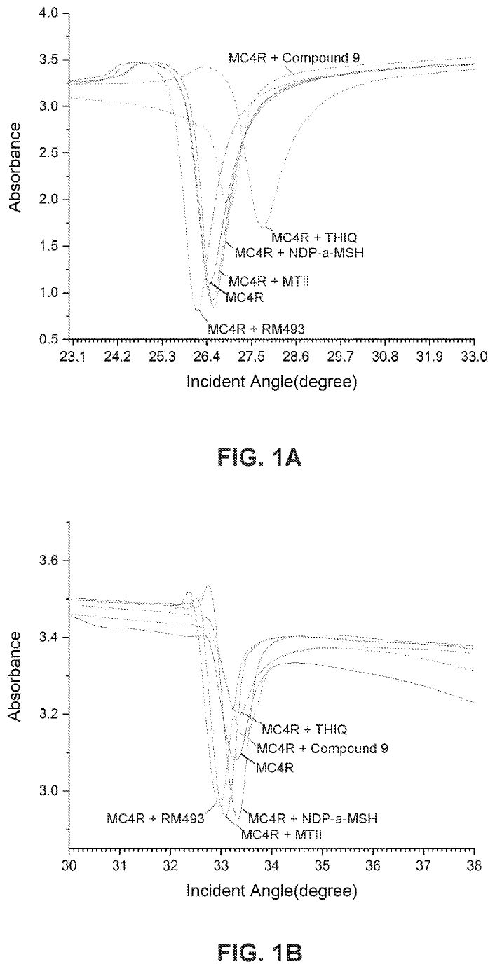

PWR spectrum absorbance of the multiple compounds binding specifically to hMC4R is shown in FIGS. 1A and 1B. In the spectrum under p-polarization, both NDP-α-MSH and MTII right shifted about 0.2 degrees (NDP-α-MSH: 26.59, MTII: 26.51) to hMC4R sole (26.44), indicating a minor size extension along MC4R long axis (perpendicular to prism surface). However, as a potentially biased Gaq agonist, RM493 left shifted 0.25 degrees compared to hMC4R sole, which means a shortened length along MC4R long axis. Under s-polarization, the NDP-α-MSH has almost the same absorbance location as sole MC4R at 33.39 and 33.29 respectively. Both RM493 and MTII shifted left at 32.97 and 31.0 specifically, compared to hMC4R with no ligands. However, very different from these 3 peptides, the THIQ, as a potential Gas biased small molecule agonist, makes the activated hMC4R conformation very different with a right shifting of the spectrum 1.35 degrees while not shifting under s-polarization. Thus, It is clear that all 3 peptide agonists have similar peak depth at around 0.8 under p-polarization and 2.95 under s-polarization, meaning similar overall MC4R+ agonists inner constructures. However, for THIQ, the peak depth is much shallower at 1.75 under p-polarization and 3.2 under s-polarization, indicating a large difference in binding and activating mechanism. THIQ is specially recognized by MC4R with very little interaction to calcium moiety and much less interactions to the reserved amino acids in MC4R binding pocket while the other 3 (e.g., NDP-α-MSH, MTII and Setmelanotide) are pretty much alike. Interestingly, the peak patterns for those 3 peptides are similar under both p- and s-polarization and at the same time, as a peptide ligand made from MTII template, compound 9 PWR spectra is very similar to the THIQ. This commonality comes from the peak pattern (similarly shallow under p- and s-polarization) and peak location (largely right shifted to 26.95 under P-polarization). Combined with observations above, it means perpendicularly, THIQ and compound 9 both lengthen the MC4R in a similar way and horizontally makes no significant change, and the very similar peak pattern gives indication of a possible similar binding mode. Even though all 5 compounds vary largely in size and molecular weight, commonalities and differences can be divided into groups, reflecting whether the properties of overall shape and internal molecular arrangement of the bulk are similar or not.

Molecular dynamics (MD) Simulation

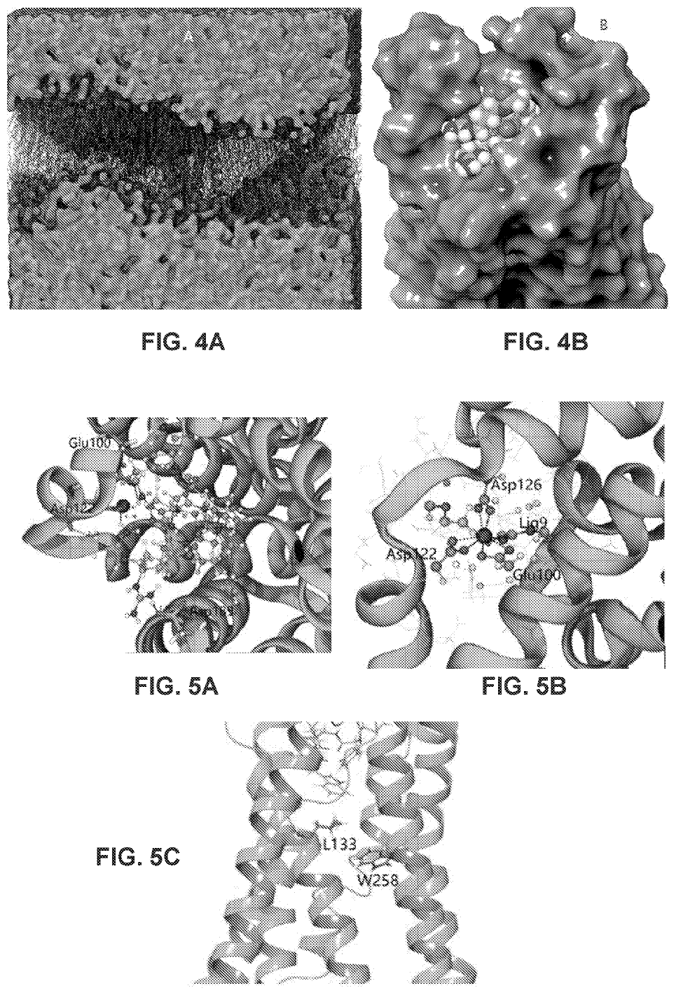

To reveal the special binding interactions of analogue 9 to hMC4R, molecular dynamics (MD) simulation was carried out. This is because of the property of MCR which belongs to GPCR and owns highly dynamic conformational change upon binding with ligands. The average structure of analogue 9 bound to hMC4R over 10 ns production run is shown in FIGS. 4A and 4B. The whole membrane system naturally turned to wave-like with up and downside filled with water and MC4R incorporated in the middle of POPC bilayer.

There are 57 charged or aromatic amino acid residues located in transmembrane (TM) regions. Among those, there are 29 conserved amino acids which represent the most important or basic function of MC4R, including Phe51 and Glu61, Asn62 in TM1; Met79, Try80, Phe82, Asp90, Glu100 in TM2; Asp122, Asp126, Asp146, Arg147, Try148 in TM3; Trp174 in TM4; Phe201, Met204, Try212, His214 and Met215 in TM5; Thr246, Thr248, Phe254, Trp258, Phe261, His264 in TM6; Phe284, Asn294, Asp298 and Tyr302 in TM7. Furthermore, alanine scan among these conserved amino acids showed that mutations of Asp122, Asp126 of TM3 and Phe 261, His264 of TM6 decreased NDP-α-MSH binding affinity. Apart from these, D90 Å, E100A and D298A also significantly altered NDP-α-MSH binding affinity. In FIGS. 5A, 5B, and 5C, it has been clearly shown that analogue 9 interacts with hMC4R through canonical conserved amino acids including Asp122, Asp126 and Glu100, which are all essential activating sites. Apart from that, analogue 9 applied similar binding posture as NDP-α-MSH, MTII and Setmelanotide that the Trp and D-Phe(4-CI) part deeply inserted into the binding pocket, reaching the wobble switch of L133 and W258 that play key role in the receptor activation. Similar to them, analogue 9 keeps the L133 up and W258 down so that TM6 can be opened a bit for G protein binding. However, what is interesting is that analogue 9 does not form as many interactions with MC4R as the 3 peptides mentioned above, nor does it form multiple electrostatic interactions with calcium, which in turn behaves as a binding mechanism more like THIQ. THIQ forms only 1 interaction with Ca2+ which is the same as analogue 9 here, showing a less dependence of metal ion when binding with MC4R. This commonality in binding mechanism may explain the similarity of peak pattern and location shifts in PWR spectra between analogue 9 and THIQ. The similar binding and activating mechanism may indicate analogue 9 taking effects like THIQ but as peptide agonist, there also might be its specialty.

### NMR Spectroscopy

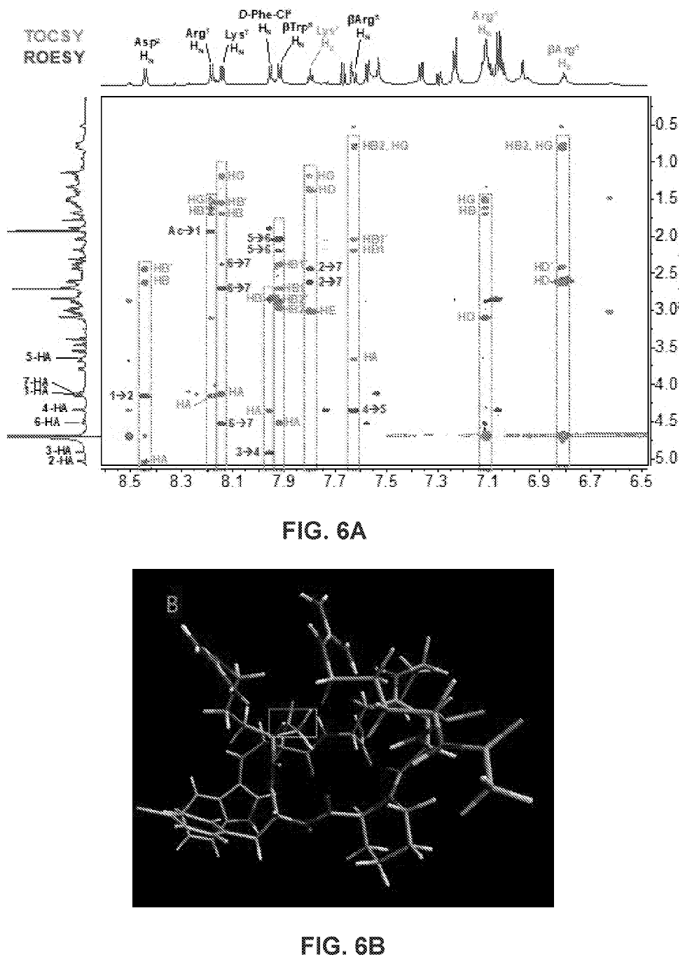

NMR spectroscopy was used to probe for structural features of the peptide observed in aqueous solution. The amide resonances were assigned using TOCSY and ROESY (FIGS. 6A and 6B), along with the sidechain resonances. Variable-temperature analysis of the H_{N} chemical shifts show relatively large 8-9 ppb/K upfield shifts with increasing temperature for all amide signals, indicating a high degree of proton exchange with the solvent. Residue 4 has the smallest H_{N} temperature coefficient of 7.5 ppb/K upfield shift, though it is still not indicative of a hydrogen bond. ³J_{HN-HA} is 7.0-7.7 Hz for residues 1, 2 and 7—typical for a random coil. For residues 5 and 6 this coupling is 9.2 Hz, which might suggest an extended conformation for these residues because of the extra methylene groups. Sequential HN(i)-HA(i-1) NOEs and ROEs are slightly stronger for residues 2, 4 and 5 than for residues 1 and 7. H_{N}-H_{N} NOEs are not observed, but conformational exchange due to Pip3 is noticeable near the diagonal in the amide region of ROESY, as seen by crosspeaks with the same phase as the diagonal. The slow rotamer exchange due to Pip3 leads to minor peaks observed for most residues that were quite different from their major counterparts, though these were excluded from analysis. The backbone H_{N} doublets are sharp for all residues except for residue 5—which could be due to some us-ms exchange broadening. The chemical shift of HA protons in residues 5 and 6 is expected to be at least 1 ppm upfield of a natural amino acid due to the extra beta-methylene group—this is observed for residue 5 but not 6, suggesting that HA of residue 6 is shifted downfield due to other structural features. It is worth noting that there are many irregular features present in this peptide for example: cyclization at side chains of residues 2 and 7, slow cis-trans isomerization of residue 3, D-amino acid at residue 4, extra beta-methylene groups in residues 5 and 6. Preliminary molecular dynamics calculations without NMR input show that the angle between HN and HA protons in residue 5 is close to zero degrees (FIGS. 6A and 6B), which is surprising but that would be consistent with HN(6)-HA(5) NOE/ROE not being observed at all. It would also be consistent with the relatively large J_{HN-HA} coupling observed for residue 5.

TABLE 4
Observed NMR parameters for backbone amide protons.

| | | | D- | | | |
|---|---|---|---|---|---|---|
| | Arg¹ | Asp² | Phe-Cl⁴ | βArg⁵ | BTrp⁶ | Lys⁷ |
| | H_{N} | H_{N} | H_{N} | H_{N} | H_{N} | H_{N} |
| ³J_{HN(i)-HA(i)} (HZ) | 7.0 | 7.7 | 7.3 | 9.2 | 9.2 | 7.5 |
| HN₍ᵢ₎-HA₍ᵢ₎ NOE | 0.3 | 1.0 | 0.6 | 0.9 | not obs. | 0.4 |
| (normalized vol.) | | | | | | |
| Linewidth (Hz) | 2.4 | 2.8 | 2.5 | 3.6 | 2.7 | 2.8 |
| Temp. coeff. (-ppB/K) | 7.8 | 8.9 | 7.5 | 8.5 | 8.5 | 8.4 |

It is worth noting that there are many irregular features present in this peptide: Cyclization at side chains of residues 2 and 7, slow cis-trans isomerization of residue 3, D-amino acid at residue 4, extra beta-methylene groups in residues 5 and 6, which would require further investigations before reliable 3D structures can be proposed. Overall, the structure appears dynamic, but it appears that there might be structural features near residue 5 that are important.

Thus, described herein the application of beta homo amino acid and para halogenation of D-Phe site, as well as the adjustment of charge through introducing more arginines makes MC4R selective agonist. Apart from the binding specificity on MC4R, analogue 9 also showed special binding and activating mechanisms which rely little on calcium and has only few interactions with several key conserved amino acids on MC4R. The special binding and activating pattern may account for the special MC4R conformation induced after analogue 9's binding.

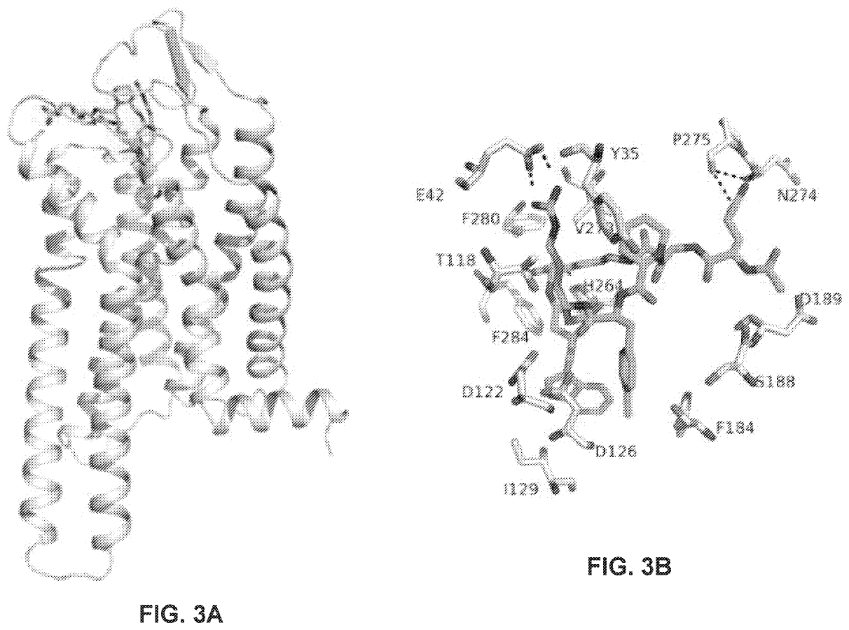

Referring to FIGS. 3A and 3B, conformational studies show that the cyclized compound 9 keeps a β-turn like structure in the binding site. Molecular docking studies indicated that the Asp 122, Asp126, and Phe 280, Phe 284 of the MC4R are the major binding sites. (Docking score 12) with compound 9.

Thus, without wishing to limit the present invention to a particular theory or mechanism, the inventors have found that the extra Arg does improve the MC4R selectivity and potency. Furthermore, the inventors have determined that compound 9 is a selective, potent agonist for the hMC4R.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A peptide according to any one of the following sequences:
(SEQ ID NO: 1)
Ac-Arg-cyclo[Asp-Pip-D-Phe-Arg-BTrp-Lys]-NH2;
(SEQ ID NO: 2)
Ac-Arg-cyclo[Asp-Pip-D-Phe-βArg-Trp-Lys]-NH2;
(SEQ ID NO: 3)
Ac-Arg-cyclo[Asp-Pip-D-Phe-βArg-BTrp-Lys]-NH2;
(SEQ ID NO: 4)
Ac-Arg-cyclo[Asp-Pip-D-Phe(4-F)-Arg-BTrp-Lys]-
NH2;
(SEQ ID NO: 5)
Ac-Arg-cyclo[Asp-Pip-D-Phe-(4-F)-βArg-Trp-Lys]-
NH2;
(SEQ ID NO: 6)
Ac-Arg-cyclo[Asp-Pip-D-Phe-(4-F)-βArg-BTrp-Lys]-
NH2;
(SEQ ID NO: 7)
Ac-Arg-cyclo[Asp-Pip-D-Phe-(4-C1)-Arg-BTrp-Lys]-
NH2;
(SEQ ID NO: 8)
Ac-Arg-cyclo[Asp-Pip-D-Phe-(4-Cl)-βArg-Trp-Lys]-
NH2;
or
(SEQ ID NO: 9)
Ac-Arg-cyclo[Asp-Pip-D-Phe-(4-C1)-βArg-BTrp-Lys]-
NH2.

2.-8. (canceled)

9. A method of treating an eating disorder in a subject in need of such treatment, the method comprising administering to the subject a pharmaceutical composition comprising a therapeutically effective amount of a peptide, wherein the peptide is a peptide agonist of melanocortin 4 receptor (MC4R), wherein the peptide is according to the following sequence:
(SEQ ID NO: 9)
Ac-Arg-cyclo[Asp-Pip-D-Phe-(4-Cl)-βArg-BTrp-Lys]-
NH2.

10. The method of claim 9, wherein the eating disorder is obesity.

11. The method of claim 9, wherein the subject is overweight or obese.

12.-15. (canceled)

16. A method of stimulating or increasing neuromelanin production in a subject in need thereof, the method comprising administering to the subject a pharmaceutical composition comprising a therapeutically effective amount of a peptide, wherein the peptide is a peptide agonist of melanocortin 4 receptor (MC4R), wherein the peptide is according to the following sequence:
(SEQ ID NO: 9)
Ac-Arg-cyclo[Asp-Pip-D-Phe-(4-C1)-βArg-BTrp-Lys]-
NH2.

17. The method of claim 16, wherein the subject is a mammal.

18. The method of claim 17, wherein the mammal is a human.

19. The method of claim 16, wherein the peptide is administered in a dosage of about 0.001 mg/kg to 100 mg/kg of body weight.

20. The method of claim 16, wherein the composition is administered at least once daily, weekly, or monthly.

21. The method of claim 16, wherein the composition is administered intranasally, intravenously, subcutaneously, transdermally, or orally.

22. (canceled)

23. (canceled)

24. A pharmaceutical composition comprising a peptide agonist of melanocortin 4 receptor (MC4R) in a pharmaceutically acceptable carrier, wherein the peptide is according to claim 1.

25. The composition of claim 24, wherein the peptide is according to SEQ ID NO: 9.

26. The composition of claim 24, wherein the pharmaceutical composition is effective for weight management or preventing or treating obesity or excess weight.

27. The composition of claim 24, wherein the pharmaceutical composition is effective for preventing and treating a neurodegenerative disease.

28. The composition of claim 27, wherein the neurodegenerative disease is Alzheimer's disease, dementia, or other memory disorders, ataxia, amyotrophic lateral sclerosis, multiple sclerosis, Huntington's disease, Parkinson's disease, motor neuron disease, multiple system atrophy, or progressive supranuclear palsy.

29. The composition of claim 24, wherein the pharmaceutical composition is effective for stimulating or increasing neuromelanin production.


SEQUENCE LISTING (10 sequences)

SEQ ID NO: 1 - protein, 7 aa, synthetic construct
  MOD_RES 3: note = pipecolic acid
  MOD_RES 1: note = acetylation
  MOD_RES 7: note = amidation
  MOD_RES 4: note = D-Phenylalanine
  MOD_RES 6: note = beta-tryptophan
  REGION 2..7: note = circular
RDXFRYK

SEQ ID NO: 2 - protein, 7 aa, synthetic construct
  MOD_RES 1: note = acetylation
  MOD_RES 7: note = amidation
  MOD_RES 3: note = pipecolic acid
  MOD_RES 5: note = beta-arginine
  REGION 2..7: note = circular
  MOD_RES 4: note = D-Phenylalanine
RDXFRYK

SEQ ID NO: 3 - protein, 7 aa, synthetic construct
  MOD_RES 1: note = acetylation
  MOD_RES 3: note = pipecolic acid
  MOD_RES 4: note = D-Phenylalanine
  MOD_RES 5: note = beta-arginine
  MOD_RES 6: note = beta-tryptophan
  MOD_RES 7: note = amidation
  REGION 2..7: note = circular
RDXFRYK

SEQ ID NO: 4 - protein, 7 aa, synthetic construct
  MOD_RES 1: note = acetylation
  MOD_RES 3: note = pipecolic acid
  MOD_RES 4: note = 4-Fluoro-D-phenylalanine
  MOD_RES 6: note = beta-tryptophan
  MOD_RES 7: note = amidation
  REGION 2..7: note = circular
RDXFRYK

SEQ ID NO: 5 - protein, 7 aa, synthetic construct
  MOD_RES 1: note = acetylation
  MOD_RES 3: note = pipecolic acid
  MOD_RES 4: note = 4-Fluoro-D-phenylalanine
  MOD_RES 5: note = beta-arginine
  MOD_RES 7: note = amidation
  REGION 2..7: note = circular
RDXFRYK

SEQ ID NO: 6 - protein, 7 aa, synthetic construct
  MOD_RES 1: note = acetylation
  MOD_RES 3: note = pipecolic acid
  MOD_RES 4: note = 4-Fluoro-D-phenylalanine
  MOD_RES 5: note = beta-arginine
  MOD_RES 6: note = beta-tryptophan
  MOD_RES 7: note = amidation
  REGION 2..7: note = circular
RDXFRYK

SEQ ID NO: 7 - protein, 7 aa, synthetic construct
  MOD_RES 1: note = acetylation
  MOD_RES 3: note = pipecolic acid
  MOD_RES 4: note = 4-Chloro-D-Phenylalanine
  MOD_RES 6: note = beta-tryptophan
  MOD_RES 7: note = amidation
  REGION 2..7: note = circular
RDXFRYK

SEQ ID NO: 8 - protein, 7 aa, synthetic construct
  MOD_RES 1: note = acetylation
  MOD_RES 3: note = pipecolic acid
  MOD_RES 4: note = 4-Chloro-D-Phenylalanine
  MOD_RES 5: note = beta-arginine
  MOD_RES 7: note = amidation
  REGION 2..7: note = circular
RDXFRYK

SEQ ID NO: 9 - protein, 7 aa, synthetic construct
  MOD_RES 1: note = acetylation
  MOD_RES 3: note = pipecolic acid
  MOD_RES 4: note = 4-Chloro-D-Phenylalanine
  MOD_RES 5: note = beta-arginine
  MOD_RES 6: note = beta-tryptophan
  MOD_RES 7: note = amidation
  REGION 2..7: note = circular
RDXFRYK

SEQ ID NO: 10 - protein, 7 aa, synthetic construct
  MOD_RES 1: note = acetylation
  MOD_RES 4: note = D-Phenylalanine
  MOD_RES 7: note = amidation
  REGION 2..7: note = circular
LDHFRYK